SEQUENCE LISTING (7 sequences)

SEQ ID NO: 1 - DNA, 33 bp, Artificial
  olignucleotide primer for PCR amplification of CHS/FLC fragment
ctcgagtctagaggagcagaagctgagatggag

SEQ ID NO: 2 - DNA, 27 bp, Artificial
  olignucleotide primer for PCR amplification of CHS/FLC fragment
ctgcaggaataaggtacaaagttcatc

SEQ ID NO: 3 - DNA, 27 bp, Artificial
  olignucleotide primer for PCR amplification of CHS/FLC fragment
ctgcaggcactgctaaccctgagaaac

SEQ ID NO: 4 - DNA, 27 bp, Artificial
  olignucleotide primer for PCR amplification of CHS/FLC fragment
ggtaccttgacttgggctggccccact

SEQ ID NO: 5 - DNA, 29 bp, Artificial
  attB1 recombination site
ggggacaagtttgtacaaaaaagcaggct

SEQ ID NO: 6 - DNA, 27 bp, Artificial
  attB2 recombination site
ggaccactttgtacaagaaagctgggt

SEQ ID NO: 7 - DNA, 17476 bp, Artificial
  plasmid pHELLSGATE 8
ggccgcactagtgatatcccgcggccatggcggccgggagcatgcgacgtcgggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaaacgttaatgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaacatcgaactacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaattaattccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaacctcgcgcatacagccgggcagtgacgtcatcgtctgcgcggaaatggacgaacagtggggctatgtcggggctaaatcgcgccagcgctggctgttttacgcgtatgacagtctccggaagacggttgttgcgcacgtattcggtgaacgcactatggcgacgctggggcgtcttatgagcctgctgtcaccctttgacgtggtgatatggatgacggatggctggccgctgtatgaatcccgcctgaagggaaagctgcacgtaatcagcaagcgatatacgcagcgaattgagcggcataacctgaatctgaggcagcacctggcacggctgggacggaagtcgctgtcgttctcaaaatcggtggagctgcatgacaaagtcatcgggcattatctgaacataaaacactatcaataagttggagtcattacccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccgggatggcgctaagaagctattgccgccgatcttcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatatcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaaacaagtggcagcaacggattcgcaaacctgtcacgccttttgtgccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggagcttggctgccatttttggggtgaggccgttcgcggccgaggggcgcagcccctggggggatgggaggcccgcgttagcgggccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgacatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgattttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgtacggaatgccagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgattgaaggagccactcagccccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatccaacgcgttgggagctctcccatatcgacctgcaggcggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagacaagtttgtacaaaaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtagtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttagtcgactacaggtcactaataccatctaagtagttgattcatagtgactggatatgttgtgttttacagtattatgtagtctgttttttatgcaaaatctaatttaatatattgatatttatatcattttacgtttctcgttcagctttcttgtacaaagtggtctcgaggaattcggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatttatcaataaacaaaatattaaataacaagctaaagtaacaaataatatcaaactaatagaaacagtaatctaatgtaacaaaacataatctaatgctaatataacaaagcgcaagatctatcattttatatagtattattttcaatcaacattcttattaatttctaaataatacttgtagttttattaacttctaaatggattgactattaattaaatgaattagtcgaacatgaataaacaaggtaacatgatagatcatgtcattgtgttatcattgatcttacatttggattgattacagttgggaagctgggttcgaaatcgataagcttggatcctctagaccactttgtacaagaaagctgaacgagaaacgtaaaatgatataaatatcaatatattaaattagattttgcataaaaaacagactacataatactgtaaaacacaacatatccagtcactatgaatcaactacttagatggtattagtgacctgtagtcgactaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttctgttcttgatgcagatgattttcaggactatgacactagcgtatatgaataggtagatgtttttattttgtcacacaaaaaagaggctcgcacctctttttcttatttctttttatgatttaatacggcattgaggacaatagcgagtaggctggatacgacgattccgtttgagaagaacatttggaaggctgtcggtcgactaagttggcagcatcacccgaagaacatttggaaggctgtcggtcgactacaggtcactaataccatctaagtagttgattcatagtgactggatatgttgtgttttacagtattatgtagtctgttttttatgcaaaatctaatttaatatattgatatttatatcattttacgtttctcgttcagcttttttgtacaaacttgtctagagtcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaaggccccaggggctagtatctacgacacaccgagcggcgaactaataacgttcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggacctgcaggcatgcaagctagcttactagtgatgcatattctatagtgtcacctaaatctgc

(19) United States
(12) Patent Application Publication
(10) Pub. No.: US 2003/0175783 A1
(43) Pub. Date: Sep. 18, 2003

(54) Methods and means for monitoring and modulating gene silencing

(76) Inventors: Peter Waterhouse, Canberra (AU); Susan Wesley, Canberra (AU); Chris Helliwell, O'Connor (AU)

Correspondence Address: BURNS DOANE SWECKER & MATHIS L L P, POST OFFICE BOX 1404, ALEXANDRIA, VA, 22313-1404, US

(21) Appl. No.: 10/385,546

(22) Filed: Mar. 12, 2003

Related U.S. Application Data
(60) Provisional application No. 60/363,852, filed on Mar. 14, 2002.

(51) Int. Cl. C12Q 1/68; A01K 67/00; C07H 21/04; C12N 1/16; C12N 1/18; A01H 1/00; C12N 15/82

(52) U.S. Cl. 435/6; 800/8; 800/288; 435/254.1; 435/254.2; 536/23.2

(57) ABSTRACT

Methods and means are provided for monitoring and modulating reduction of gene expression in eukaryotic organisms, using double-stranded RNA comprising, in addition to the dsRNA region comprising nucleotide sequences homologous to the target gene, additional dsRNA regions designed to down regulate a second gene or which are unrelated to the target gene.

### FIELD OF THE INVENTION

The present invention relates to methods of altering the expression of genes in eukaryotic organisms, such as plants, but also animals such as nematodes, insects and arthropods, mammals including humans, or yeasts, fungi or molds, using dsRNA capable of altering the expression of target genes, or genes encoding such dsRNA. Also provided are eukaryotic organisms comprising such dsRNA, and genes encoding such dsRNA.

In a first aspect, the invention provides methods and means for monitoring the silencing of a target gene in a eukaryotic cell by measuring the degree of silencing of a second gene wherein the silencing of the target gene and of the second gene is obtained through the action of a single initial dsRNA molecule provided to the eukaryotic cell.

In a second aspect, the invention provides methods and means for modulating the degree of silencing of a target gene in a eukaryotic cell. These methods and means comprise including into the single initial dsRNA molecule provided to the eukaryotic cell the target dsRNA inducing the silencing of the target gene, and an amount of dsRNA sequences unrelated to the target dsRNA, in a proportion reflecting the desired modulation of the degree of silencing of the target gene.

### BACKGROUND ART

Until recently, two predominant methods for the modulation of gene expression in eukaryotic organisms were known, which are referred to in the art as “antisense” downregulation and “sense” downregulation.

Recent work has demonstrated that gene silencing efficiency could be greatly improved both on quantitative and qualitative level using chimeric constructs encoding RNA capable of forming a double-stranded RNA by base pairing, between the antisense and sense RNA, nucleotide sequences respectively complementary and homologous to the target sequences. Such double-stranded RNA (dsRNA) is also referred to as hairpin RNA (hpRNA) or interfering RNA (RNAi).

Downregulating the expression of target nucleotide sequence using dsRNA has been described as a convenient analysis method for the elucidation of the function of nucleic acids (see e.g. WO99/53050 or WO00/0846). dsRNA mediated gene silencing can also be used to modulate the expression of one or more target genes in an organism to obtain a modified organism with a desired phenotype or trait (WO99/53050).

It would be helpful for the above-mentioned applications to have a way for easily monitoring the quantitative and qualitative downregulation of the target nucleic acids. This is particularly true where determination of the downregulation of the expression of the target gene through the analysis of the phenotype caused by the downregulation is labor-intensive, cost-intensive, time-consuming, requires the use of specialized analysis methods, etc. When dsRNA-mediated gene silencing is used for function identification/validation of unknown genes, where by definition the phenotype to look for is not known, it is imperative to have an alternative method for monitoring the efficiency of the silencing.

Further, it would be convenient to be able to modulate the degree of silencing of a particular target gene or genes in an organism, e.g. by generating a population of organisms exhibiting a large spectrum in the individual degrees of dsRNA-mediated gene silencing, and identifying the organisms with the desired degree of silencing.

Fire et al., 1998 describe specific genetic interference by experimental introduction of double-stranded RNA in Caenorhabditis elegans.

WO 99/32619 provides a process of introducing RNA into a living cell to inhibit gene expression of a target gene in that cell. The process may be practiced ex vivo or in vivo. The RNA has a region with double-stranded structure. Inhibition is sequence-specific in that the nucleotide sequences of the duplex region of the RNA and/or a portion of the target gene are identical.

Waterhouse et al. 1998 describes that virus resistance and gene silencing in plants can be induced by simultaneous expression of sense and antisense RNA. The sense and antisense RNA may be located in one transcript that has self-complementarity.

Hamilton et al. 1998 describes that a transgene with repeated DNA, i.e., inverted copies of its 5′ untranslated region, causes high frequency, post-transcriptional suppression of ACC-oxidase expression in tomato.

WO 98/53083 describes constructs and methods for enhancing the inhibition of a target gene within an organism which involve inserting into the gene silencing vector an inverted repeat sequence of all or part of a polynucleotide region within the vector.

WO 99/53050 provides methods and means for reducing the phenotypic expression of a nucleic acid of interest in eukaryotic cells, particularly in plant cells, by introducing chimeric genes encoding sense and antisense RNA molecules directed towards the target nucleic acid. These molecules are capable of forming a double-stranded RNA region by base pairing between the regions with the sense and antisense nucleotide sequence or by introducing the RNA molecules themselves. The RNA molecules may comprise simultaneously both sense and antisense nucleotide sequences.

WO 99/49029 relates generally to a method of modifying gene expression, and to synthetic genes for modifying endogenous gene expression in a cell, tissue or organ of a transgenic organism, in particular to a transgenic animal or plant. Synthetic genes and genetic constructs, capable of forming a dsRNA which is capable of repressing, delaying or otherwise reducing the expression of an endogenous gene or a target gene in an organism when introduced thereto are also provided.

WO 99/61631 relates to methods to alter the expression of a target gene in a plant using sense and antisense RNA fragments of the gene. The sense and antisense RNA fragments are capable of pairing and forming a double-stranded RNA molecule, thereby altering the expression of the gene. The present invention also relates to plants, their progeny and seeds thereof obtained using these methods.

WO 00/01846 provides a method of identifying DNA responsible for conferring a particular phenotype in a cell. This method comprises: (a) constructing a cDNA or genomic library of the DNA of the cell in a suitable vector in an orientation relative to one or more promoters capable of initiating transcription of the cDNA or DNA to double-stranded (“ds”) RNA upon binding of an appropriate transcription factor to the promoter(s); (b) introducing the library into one or more cells comprising the transcription factor; and (c) identifying and isolating a particular phenotype of a cell comprising the library and identifying the DNA or cDNA fragment from the library responsible for conferring the phenotype. Using this technique, it is also possible to assign function to a known DNA sequence by (a) identifying homologues of the DNA sequence in a cell; (b) isolating the relevant DNA homologue(s) or a fragment thereof from the cell; (c) cloning the homologue(s) or fragment(s) thereof into an appropriate vector in an orientation relative to a suitable promoter capable of initiating transcription of dsRNA from the DNA homologue(s) or fragment(s) upon binding of an appropriate transcription factor to the promoter; and (d) introducing the vector into the cell from step (a) comprising the transcription factor.

WO 00/44914 also describes composition and methods for in vivo and in vitro attenuation of gene expression using double-stranded RNA, particularly in zebrafish.

WO 00/49035 discloses a method for silencing the expression of an endogenous gene in a cell. The method involves overexpressing in the cell a nucleic acid molecule of the endogenous gene and an antisense molecule including a nucleic acid molecule complementary to the nucleic acid molecule of the endogenous gene, wherein the overexpression of the nucleic acid molecule of the endogenous gene and the antisense molecule in the cell silences the expression of the endogenous gene.

Smith et al., 2000 as well as WO 99/53050 described that intron-containing dsRNA further increased the efficiency of silencing. Intron-containing hairpin RNA is often also referred to as ihpRNA.

As illustrated by the above-mentioned references, dsRNA-mediated gene silencing is a phenomenon that occurs in a wide range of eukaryotic organisms, including plants, yeasts or fungi, insects, arthropods and vertebrate animals, including mammals.

WO 93/23551 describes a process for the inhibition of two or more target genes. This process comprises introducing into a plant a single control gene, which has distinct DNA regions homologous to each of the target genes, and a promoter operative in plants adapted to transcribe from such distinct regions either antisense or sense RNA that inhibits expression of each of the target genes.

WO 99/49029 describes a method for simultaneously targeting the expression of several target genes which are co-expressed in a particular cell, for example by using a dispersed nucleic acid molecule or foreign nucleic acid molecule which comprises nucleotide sequences which are substantially identical to each of the co-expressed target genes.

However, none of the above-mentioned prior art references has addressed the problem of monitoring the degree of silencing of a target gene with a phenotype that is difficult or practically impossible to measure. The prior art also is deficient, as it does not provide a method for modulating or fine-tuning the degree of silencing of a specific target gene or genes using dsRNA.

These and other problems have been solved by the present inventors as described hereinafter in the different embodiments and claims.

### SUMMARY OF THE INVENTION

The invention provides a method for monitoring the reduction of the expression of a target gene in a cell of a eukaryotic organism, such as a plant, animal, yeast, fungus or mold, comprising the steps of:

providing the eukaryotic cell with a dsRNA comprising a first region, a second region, a third region and a fourth region, wherein

the first region comprises a nucleotide sequence of at least about 19 consecutive nucleotides, which has at least about about 94% sequence identity to about 19 consecutive nucleotides from a sense nucleotide region of the target gene;

the second region comprises a nucleotide sequence of at least about 19 consecutive nucleotides, which has at least about about 94% sequence identity to a nucleotide sequence complementary to about 19 consecutive nucleotides from the sense nucleotide region of the target gene;

the first region and the second region are capable of forming a double-stranded RNA region;

the third region comprises a nucleotide sequence of at least about 19 consecutive nucleotides, which has at least about about 94% sequence identity to about 19 consecutive nucleotides from a sense nucleotide region of a second gene, such as an endogenous gene or a transgene, stably integrated into the genome of the eukaryotic cell and which is different from the target gene;

the fourth region comprises a nucleotide sequence of at least about 19 consecutive nucleotides, which has at least about about 94% sequence identity to the complement of the about 19 consecutive nucleotides from the sense nucleotide region of the second gene;

the third and fourth regions are capable of forming a double-stranded RNA region; and

monitoring the reduction of the expression of the target gene by analyzing the reduction in expression of the second gene.

The dsRNA may be transcribed from a chimeric gene comprised within cells of the eukaryotic organism, wherein the chimeric gene comprises the following operably linked elements:

a promoter region that functions in the eukaryotic cell,

a DNA region which, when transcribed, yields the dsRNA molecule; and

a transcription termination and polyadenylation region that functions in the cells of eukaryotic organism.

It is also an object of the invention to provide an RNA molecule as described above, as well as the use of such an RNA molecule for measuring the reduction of expression of a target gene.

It is another object of the invention to provide a DNA molecule for measuring the reduction of expression of a target gene in a eukaryotic cell, wherein the DNA molecule comprises the following operably linked elements:

a promoter region that functions in the eukaryotic cell;

a DNA region which, when transcribed, yields a dsRNA molecule; and

a transcription termination and polyadenylation region that functions in cells of the eukaryotic organism;

wherein the dsRNA comprises a first, second, third and fourth region, wherein

the first region comprises a nucleotide sequence of at least about 19 consecutive nucleotides, which has at least about about 94% sequence identity to about 19 consecutive nucleotides from a sense nucleotide region of the target gene;

the second region comprises a nucleotide sequence of at least about 19 consecutive nucleotides, which has at least about about 94% sequence identity to a nucleotide sequence complementary to about 19 consecutive nucleotides from the sense nucleotide region of the target gene;

the first region and the second region are capable of forming a double-stranded RNA region;

the third region comprises a nucleotide sequence of at least about 19 consecutive nucleotides, which has at least about about 94% sequence identity to about 19 consecutive nucleotides from a sense nucleotide region of a second gene present in the eukaryotic cell and which is different from the target gene;

the fourth region comprises a nucleotide sequence of at least about 19 consecutive nucleotides, which has at least about about 94% sequence identity to the complement of the about 19 consecutive nucleotides from the sense nucleotide region of the second gene;

the third and fourth regions are capable of forming a double-stranded RNA region; and

wherein the second gene is an endogenous gene of the eukaryotic organism or a transgene stably integrated into the genome of cells of the eukaryotic organism, as well as the use of such a DNA molecule to measure the expression of a target gene by measuring the reduction in expression of a second gene.

The invention also provides eukaryotic organisms comprising an RNA molecule or a DNA molecule as herein described.

Also provided by the invention is a method for identifying, within a population of dsRNA-mediated gene-silenced eukaryotic organisms, those organisms with the desired degree of silencing of a target gene comprising:

providing cells of the eukaryotic organisms with a dsRNA comprising a first region, a second region, a third region and a fourth region, wherein

the first region comprises a nucleotide sequence of at least about 19 consecutive nucleotides, which has at least about about 94% sequence identity to about 19 consecutive nucleotides from a sense nucleotide region of the target gene;

the second region comprises a nucleotide sequence of at least about 19 consecutive nucleotides, which has at least about 94% sequence identity to a nucleotide sequence complementary to about 19 consecutive nucleotides from the sense nucleotide region of the target gene;

the first region and the second region are capable of forming a double-stranded RNA region;

the third region comprises a nucleotide sequence of at least about 19 consecutive nucleotides, which has at least about 94% sequence identity to about 19 consecutive nucleotides from a sense nucleotide region of a second gene present in the eukaryotic cells, and which is different from the target gene;

the fourth region comprises a nucleotide sequence of at least about 19 consecutive nucleotides having at least about 94% sequence identity to the complement of the about 19 consecutive nucleotides from the sense nucleotide region of the second gene;

the third and fourth regions are capable of forming a double-stranded RNA region; and

identifying the organism with the desired degree of silencing of the target gene, by selecting those organisms with the desired degree of silencing of the second gene.

It is a further object of the invention to provide a method for modulating the reduction of the expression of a target gene in cells of a eukaryotic organism, comprising the steps of:

providing the eukaryotic cell with a dsRNA comprising a first region, a second region, a third region and a fourth region, wherein

the first region comprises a nucleotide sequence of at least about 19 consecutive nucleotides, which has at least about 94% sequence identity to about 19 consecutive nucleotides from the sense nucleotide sequence of the target gene;

the second region comprises a nucleotide sequence of at least about 19 consecutive nucleotides, which has at least about 94% sequence identity to the complement of about 19 consecutive nucleotides from the sense nucleotide sequence of the target gene;

the first region and the second region are capable of forming a double-stranded RNA region;

the third region and the fourth region comprise complementary nucleotide sequences, which have a sequence identity of less than 50% to the nucleotide sequence of the target gene, and which are capable of forming a double-stranded RNA;

wherein the target gene is an endogenous gene in the eukaryotic cell or a transgene stably integrated in the genome of the eukaryotic cell.

The size of the double-stranded RNA capable of being formed by base pairing between the third and fourth region may be equal or larger than the size of the double-stranded RNA capable of being formed by base pairing between the first and the second region.

The invention also provides dsRNA molecules suitable for the modulation of the degree of expression of a target gene in cells of eukaryotic organism, chimeric genes capable of yielding such dsRNA molecules, and organisms containing such dsRNA molecules or chimeric genes.

### BRIEF DESCRIPTION OF THE FIGURES

FIG. 1 shows seeds from T1 plants transformed with the FLC/CHS construct, fluorescing under UV light, compared to seeds from C24 wildtype (C24), a C24 wildtype containing a chimeric GUS gene under control of a CaMV35S promoter (GUS) and a homozygous CHS silenced line (CHS). Numbers indicate the days to flowering of the transgenic plant cell lines.

FIG. 2 shows a Northern blot analysis of RNA prepared from transgenic plant lines, comprising dsRNA encoding genes targeted towards both FLC and CHS, probed with an FLC probe (upper panel) and a CHS probe (lower panel) (FIG. 2A). FIG. 2B is a graphic representation of the the amount of mRNA detected from FLC expression plotted against the amount of mRNA detected from CHS expression.

### DETAILED DESCRIPTION OF EMBODIMENTS OF THE INVENTION

The invention is based on the unexpected observation that when a eukaryotic cell, such as a plant cell, comprises a double-stranded RNA (dsRNA) wherein the dsRNA comprises simultaneously complementary antisense and sense regions for at least two target genes, a correspondence exists between the degree of silencing of expression of all of the targeted genes. The correspondence further depends upon the relative size of the antisense and sense regions designed to reduce the expression of the different target genes.

This correspondence may conveniently be used to monitor silencing of genes, the expression of which results in a phenotype that is not straightforward to monitor. This may be accomplished by linking the complementary sense and antisense regions suitable to reduce or silence the expression of such a gene, to complementary sense and antisense regions suitable for reducing or silencing the expression of a gene whose expression results in a phenotype which can be monitored in a straightforward way.

Thus, in one embodiment of the invention, a method is provided for monitoring the reduction or silencing of the expression of a target gene in a eukaryotic cell, comprising the steps of:

providing the eukaryotic cell with a dsRNA comprising a first region, a second region, a third region and a fourth region, wherein

the first region comprises a nucleotide sequence of at least about 19 consecutive nucleotides having at least about 94% sequence identity to about 19 consecutive nucleotides from the sense nucleotide region of the target gene;

the second region comprises a nucleotide sequence of at least about 19 consecutive nucleotides having at least about 94% sequence identity to a nucleotide sequence complementary to about 19 consecutive nucleotides from the sense nucleotide region of the target gene;

the first region and second region are capable of forming a double-stranded RNA region, which may be over the entire length of the first and second region, and at least over the length of the mentioned about 19 nucleotides;

the third region comprises a nucleotide sequence of at least about 19 consecutive nucleotides having at least about 94% sequence identity to about 19 consecutive nucleotides from the sense nucleotide region of a second gene present in the eukaryotic cell and which is different from the target gene;

the fourth region comprises a nucleotide sequence of at least about 19 consecutive nucleotides having at least about 94% sequence identity to the complement of about 19 consecutive nucleotides from the sense nucleotide region of the same second gene;

the third and fourth region are capable of forming a double-stranded RNA region, which may be over the entire length of the first and second region, and at least over the length of the mentioned about 19 nucleotides; and

monitoring the expression of the degree of silencing of the target gene, by analyzing the reduction in expression of the second gene.

In one embodiment of the present invention, the first and second regions are capable of forming a double-stranded RNA region at the same time as when the third and fourth regions are forming a double-stranded RNA.

The second gene different from the target gene is used as a reporter gene to monitor the degree of silencing of the target gene. Whenever reference is made herein to a reporter gene, it is understood that this term is used to refer to a second gene present in the eukaryotic cell and which is different from the target gene.

For the purposes of the current invention, it is equal whether that second gene is an endogene, normally present in the eukaryotic cell, or a transgene, which has been introduced into the eukaryotic cell by human intervention at some point in history, and which may be integrated into the genome of the eukaryotic cell in a stable manner.

The reporter gene may have a phenotype, the analysis of which is more straightforward than the analysis of the target gene or genes, in terms of requirements of costs, expertise, time, labor, used apparatuses, etc.

The silencing of the reporter gene should not have a negative influence on the viability of the host cell or host organism, although the absence of negative influence may be dependent on particular conditions.

Conveniently, silencing the expression of the reporter gene may result in a visible phenotype, although the visibility of the phenotype may again be conditional.

Examples of reporter genes suitable for application of the methods of the invention in plant cells and plants include, but are not limited to: Chalcone synthase gene (CHS, where down-regulation of the expression results in accumulation of UV-fluorescent compounds in the seed coat color); phytoene desaturase gene (PDS, where down-regulation of the gene expression results in photobleaching); flower locus C (FLC, where down-regulation of the gene expression results in early flowering); ethylene insensitivity gene 2 (EIN2, where down-regulation of the gene results in plants which are insensitive to ethylene, and will grow on media containing 1-aminocyclopropane-1-carboxylic acid(AAC)); visual marker genes such as seed coat color genes (e.g., R-gene in corn), plant-expressible GUS or GFP genes, phytochrome B and the like.

Examples of reporter genes suitable for application of the methods of the invention to animal cells and animals include, but are not limited to, GUS or GFP genes operably linked to expression regions suitable for animal cells; genes such as unc in Caenorhabditis elegans, the silencing of which causes a characteristic twisting pattern in the nematodes; and the like.

Examples of reporter genes suitable for use in the methods of the invention in fungal cells include but are not limited to: GUS or GFP genes operably linked to expression regions suitable for fungus cells; and genes the silencing of which causes auxotrophic growth (such as, e.g., trpC), a phenotype which can be easily screened on minimal media In these cases, a master copy of the library of silenced fungal cells needs to be maintained under conditions allowing growth, e.g., in the presence of the required nutrient compound.

The reporter gene may be expressed under conditions where the target gene is expressed.

The about 19 nucleotides that are at least about 94% identical or complementary to a nucleotide sequence of about 19 consecutive nucleotides of the target gene and/or the reporter gene may be comprised within a larger RNA molecule. Such an RNA molecule will have a nucleotide sequence varying between as little as about 19 bp to a length equal to the size of the target gene with a varying overall degree of sequence identity.

For the purpose of this invention, the “sequence identity” of two related nucleotide or amino acid sequences, expressed as a percentage, refers to the number of positions in the two optimally aligned sequences which have identical residues (xl 00) divided by the number of positions compared. A gap, i.e., a position in an alignment where a residue is present in one sequence but not in the other, is regarded as a position with non-identical residues. The alignment of the two sequences is performed by the Needleman and Wunsch algorithm (Needleman and Wunsch about 1970). The computer-assisted sequence alignment above can be conveniently performed using standard software program such as GAP, which is part of the Wisconsin Package Version 10.1 (Genetics Computer Group, Madision, Wis., USA) using the default scoring matrix with a gap creation penalty of 50 and a gap extension penalty of 3. Sequences are indicated as “essentially similar” when such sequence have a sequence identity of at least about 75%, at least about 80%, at least about 85%, about 90%, about 95%, about 100%, or are identical. It is clear than when RNA sequences are the to be essentially similar or have a certain degree of sequence identity with DNA sequences, thymine (T) in the DNA sequence is considered equal to uracil (U) in the RNA sequence. Thus when it is stated in this application that a sequence of about 19 consecutive nucleotides has a about 94% sequence identity to a sequence of about 19 nucleotides, this means that at least about 18 of the about 19 nucleotides of the first sequence are identical to about 18 of the about 19 nucleotides of the second sequence.

The mentioned sense or antisense nucleotide regions may thus be about 50 nt, 100 nt, 200 nt, 300 nt, 500 nt, 1000 nt, 2000 nt or even about 5000 nt or longer, each having an overall sequence identity of respectively about 40%, 50%, 60%, 70%, 80%, 90% or 100%. The longer the sequence, the less stringent the requirement for the overall sequence identity.

The first, second, third and fourth region each may be separated by a spacer region having a nucleotide sequence which is unrelated to the nucleotide sequence of either the target or the reporter gene.

For the purpose of the invention, although the regions are named consecutively, the order of the dsRNA regions in the dsRNA molecule is not important. In other words, it does not matter whether, e.g., the first or second region or alternatively the third or fourth region is located at the 5′ or 3′ end of the RNA molecule.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region that is defined functionally or structurally may comprise additional DNA regions, etc.

An dsRNA molecule comprising a first, second, third and fourth region as herein defined may thus additionally include, e.g.,: a fifth and sixth region having a nucleotide region of about 19 nucleotides with at least about 94% sequence identity or complementarity to, e.g., a target gene, which may be the same target gene as the first target gene, or may be a different one.

In one embodiment of the invention, the dsRNA molecule may further comprise one or more regions having at least about 94% sequence identity to regions of about 19 consecutive nucleotides from the sense nucleotide of the target genes, different from the about 19 consecutive nucleotides as defined in the first region, and one or more regions having at least about 94% sequence identity to about 19 consecutive nucleotides from the complement of the sense nucleotide of the target gene, different from the about 19 consecutive nucleotides as defined in the second region, wherein these additional regions can basepair among themselves. Similarly, the dsRNA may additionally comprise one or more regions having at least about 94% sequence identity to regions of about 19 consecutive nucleotides of a reporter gene different from the about 19 nucleotides of the third region and one or more regions having at least about 94% sequence identity to the complementary regions of about 19 consecutive nucleotides of the reporter genes, wherein these additional regions are capable of basepairing among themselves. Again, no particular order of the regions is required, and these regions may be dispersed among each other. Thus, e.g., dsRNA regions directed towards silencing of the target gene may be alternated with dsRNA regions directed towards silencing of the reporter gene, provided that basepairing between complementary RNA regions is still possible.

Conveniently, the dsRNA as described may be introduced into the host cell by introduction and possible integration of a chimeric gene, transcription of which yields such a dsRNA. Thus the invention is also aimed at providing such a chimeric gene comprising:

a promoter or a promoter region which is capable of being expressed in cells of the eukaryotic organism of interest, operably linked to a DNA region which when transcribed yields a dsRNA molecule comprising a first region, a second region, a third region and a fourth region, wherein

the first region comprises a nucleotide sequence of at least about 19 consecutive nucleotides having at least about 94% sequence identity to about 19 consecutive nucleotides from the sense nucleotide sequence of the target gene;

the second region comprises a nucleotide sequence of at least about 19 consecutive nucleotides having at least about 94% sequence identity to the complement of about 19 consecutive nucleotides from the sense nucleotide sequence of the target gene;

the first region and second region are capable of forming a double-stranded RNA region, which may be over the entire length of the first and second region and at least between the mentioned about 19 nucleotides of the first and second region;

the third region comprises a nucleotide sequence of at least about 19 consecutive nucleotides having at least about 94% sequence identity to about 19 consecutive nucleotides from the sense nucleotide sequence of a second gene present in the eukaryotic cell and which is different from the target gene;

the fourth region comprises a nucleotide sequence of at least about 19 consecutive nucleotides having at least about 94% sequence identity to the complement of about 19 consecutive nucleotides from the sense nucleotide sequence of the same second gene;

the third and fourth region are capable of forming a double-stranded RNA region, which may be over the entire length of the first and second region, and at least over the length of the mentioned about 19 nucleotides; and

the double-stranded RNA regions formed between the first and second region and the double-stranded RNA region formed between the third and fourth region are about equal in size; and

a transcription termination and polyadenylation region suitable for the eukaryotic cell of choice.

As used herein, the term “promoter” denotes any DNA that is recognized and bound (directly or indirectly) by a DNA-dependent RNA-polymerase during initiation of transcription. A promoter includes the transcription initiation site, and binding sites for transcription initiation factors and RNA polymerase, and can comprise various other sites (e.g., enhancers) at which gene expression regulatory proteins may bind.

The term “regulatory region”, as used herein, means any DNA that is involved in driving transcription and controlling (i.e., regulating) the timing and level of transcription of a given DNA sequence, such as a DNA coding for a protein or polypeptide. For example, a “5′ regulatory region” (or “promoter region”) is a DNA sequence located upstream (i.e., 5′) of a coding sequence and which comprises the promoter and the 5′-untranslated leader sequence. A “3′ regulatory region” is a DNA sequence located downstream (i.e., 3′) of the coding sequence and which comprises suitable transcription termination (and/or regulation) signals, including one or more polyadenylation signals.

In one embodiment of the invention, the promoter is a constitutive promoter. In another embodiment of the invention, the promoter activity is enhanced by external or internal stimuli (inducible promoter) such as but not limited to hormones, chemical compounds, mechanical impulses, and abiotic or biotic stress conditions. The activity of the promoter may also be regulated in a temporal or spatial manner (e.g., tissue-specific promoters; developmentally regulated promoters).

In one embodiment of the invention, the promoter is a plant-expressible promoter. As used herein, the term “plant-expressible promoter” means a DNA sequence that is capable of initiating and/or controlling transcription in a plant cell. This includes any promoter of plant origin; any promoter of non-plant origin which is capable of directing transcription in a plant cell, e.g., certain promoters of viral or bacterial origin such as the CaMV35S (Hapster et al., 1988), the subterranean clover virus promoter No 4 or No 7 (WO9606932), and T-DNA gene promoters; tissue-specific or organ-specific promoters, including but not limited to seed-specific promoters (e.g., WO89/03887), organ-primordia specific promoters (An et al., 1996), stem-specific promoters (Keller et al., 1988), leaf-specific promoters (Hudspeth et al., 1989), mesophyl-specific promoters (such as the light-inducible Rubisco promoters), root-specific promoters (Keller et al., 1989), tuber-specific promoters (Keil et al., 1989), vascular tissue-specific promoters (Peleman et al., 1989), stamen-selective promoters (WO 89/10396, WO 92/13956), dehiscence zone-specific promoters (WO 97/13865); and the like.

In another particular embodiment of the invention, the promoter is a fungus-expressible promoter. As used herein, the term “fungus-expressible promoter” means a DNA sequence that is capable of initiating and/or controlling transcription in a fungal cell, such as but not limited to the A. nidulans trpC gene promoter, or the S. cerevisiae GAL4 promoter.

In yet another particular embodiment of the invention, the promoter is a animal-expressible promoter. As used herein, the term “animal-expressible promoter” means a DNA sequence which is capable of initiating and/or controlling transcription in an animal cell, and including but not limited to SV40 late and early promoters, cytomegalovirus CMV-IE promoters, RSV-LTR promoter, SCSV promoter, SCBV promoter and the like.

The dsRNA molecules useful for the invention may also be produced by in vitro transcription. To this end, the promoter of the chimeric genes according to the invention may be a promoter recognized by a bacteriophage single subunit RNA polymerase, such as the promoters recognized by bacteriophage single subunit RNA polymerase; the RNA polymerases derived from the E. coli phages T7, T3, φI, φII, W31, H, Y, A1, 122, cro, C21, C22, and C2; Pseudomonas putida phage gh-1; Salmonella typhimurium phage SP6; Serratia marcescens phage IV; Citrobacter phage Vilil; and Klebsiella phage No. 11 (Hausmann, Current Topics in Microbiology and Immunology, 75: 77-109 (1976); Korsten et al., J. Gen Virol. 43: 57-73 (1975); Dunn et al., Nature New Biology, 230: 94-96 (1971); Towle et al., J. Biol. Chem. 250: 1723-1733 (1975); Butler and Chamberlin, J. Biol. Chem., 257: 5772-5778 (1982)). Examples of such promoters are a T3 RNA polymerase specific promoter and a T7 RNA polymerase specific promoter, respectively. A T3 promoter to be used as a first promoter in the CIG can be any promoter of the T3 genes as described by McGraw et al, Nucl. Acid Res. 13: 6753-6766 (1985). Alternatively, a T3 promoter may be a T7 promoter that is modified at nucleotide positions −10, −11 and −12 in order to be recognized by T3 RNA polymerase (Klement et al., J. Mol. Biol. 215, 21-29(1990)). One alternative T3 promoter is the promoter having the “consensus” sequence for a T3 promoter, as described in U.S. Pat. No. 5,037,745. A T7 promoter which may be used according to the present invention, in combination with T7 RNA polymerase, comprises a promoter of one of the T7 genes as described by Dunn and Studier, J. Mol. Biol. 166: 477-535 (1983). One alternative T7 promoter is a promoter having the “consensus” sequence for a T7 promoter, as described by Dunn and Studier (supra).

dsRNA can be produced in large amounts by contacting the acceptor vector DNA with the appropriate bacteriophage single subunit RNA polymerase under conditions well known to the skilled artisan. The so-produced dsRNA can then be used for delivery into cells prone to gene silencing, such as plant cells, fungal cells or animal cells. dsRNA may be introduced in animal cells via liposomes or other transfection agents (e.g. Clonfection transfection reagent or the CalPhos Mammalian transfection kit from ClonTech) and could be used for methods of treatment of animals, including humans, by silencing the appropriate target genes. dsRNA can be introduced into the cell in a number of different ways. For example, the dsRNA may be administered by microinjection, bombardment by particles covered by the dsRNA, soaking the cell or organism in a solution of the dsRNA, electroporation of cell membranes in the presence of dsRNA, liposome-mediated delivery of dsRNA and transfection mediated by chemicals such as calcium phosphate, viral infection, transformation and the like. The dsRNA may be introduced along with components that enhance RNA uptake by the cell, stabilize the annealed strands, or otherwise increase inhibition of the target gene. In the case of a whole animal, the dsRNA is conveniently introduced by injection or perfusion into a cavity or interstitial space of an organism, or systemically via oral, topical, parenteral (including subcutaneous, intramuscular or intravenous administration), vaginal, rectal, intranasal, ophthalmic, or intraperitoneal administration. The dsRNA may also be administered via an implantable extended-release device.

The chimeric genes according to the invention capable of producing a dsRNA may also be equipped with any prokaryotic promoter suitable for expression of dsRNA in a particular prokaryotic host. The prokaryotic host can be used as a source of dsRNA, e.g. by feeding it to an animal, such as a nematode or an insect, in which the silencing of the target gene is envisioned and monitored by reduction of the expression of the reporter gene. In this case, it will be clear that the target gene and reporter genes should be genes present in the cells of the target eukaryotic organism and not genes of the prokaryotic host organism. The dsRNA according to the invention, or chimeric genes capable of yielding such dsRNA molecules, can be thus produced in one host organism. The dsRNA can be administered to another target organism (e.g. through feeding, orally administering, as a naked DNA or RNA molecule or encapsulated in a liposome, in a virus particle or attentuated virus particle, or on an inert particle etc.) and effect reduction of gene expression in the target gene or genes and reporter gene or genes in that target organism. In this case, it will be clear that the target gene and reporter genes should be genes present in the cells of the target (eukaryotic) organism and not genes of the host organism.

Suitable transcription termination and polyadenylation regions include but are not limited to the SV40 polyadenylation signal, the HSV TK polyadenylation signal, the nopaline synthase gene terminator of Agrobacterium tumefaciens, the terminator of the CaMV ³⁵S transcript, terminators of the subterranean stunt clover virus, the terminator of the Aspergillus nidulans trpC gene and the like.

The present invention also includes providing the dsRNA molecules, which may be obtained by transcription from these chimeric genes, and which are useful for the methods according to the invention.

It is another object of the invention to provide eukaryotic cells, and eukaryotic organisms containing the dsRNA molecules of the invention, or containing the chimeric genes capable of producing the dsRNA molecules of the invention. The chimeric genes may be stably integrated in the genome of the cells of the eukaryotic organism.

In another embodiment, the chimeric genes may be provided on a DNA molecule capable of autonomously replicating in the cells of the eukaryotic organism, such as e.g. viral vectors. The chimeric gene or the dsRNA may be also be provided transiently to the cells of the eukaryotic organism.

The dsRNA molecules according to this first aspect of the invention can also be used for identifying, within a population of dsRNA-mediated gene-silenced organisms, those with the desired degree of silencing of a target gene or genes. To this end, a population of dsRNA-containing organisms is generated, wherein the dsRNA comprises a first region, a second region, a third region and a fourth region, wherein the first region comprises a nucleotide sequence of at least about 19 consecutive nucleotides having at least about 94% sequence identity to about 19 consecutive nucleotides from the sense nucleotide sequence of the target gene. The second region comprises a nucleotide sequence of at least about 19 consecutive nucleotides having at least about 94% sequence identity to the complement of about 19 consecutive nucleotides from the sense nucleotide sequence of the target gene. The third region comprises a nucleotide sequence of at least about 19 consecutive nucleotides having at least about 94% sequence identity to about 19 consecutive nucleotides from the sense nucleotide sequence of a second gene present in the eukaryotic cell and which is different from the target gene. The fourth region comprises a nucleotide sequence of at least about 19 consecutive nucleotides having at least about 94% sequence identity to the complement of about 19 consecutive nucleotides from the sense nucleotide sequence of the same second gene. The first and second and the third and fourth regions are capable of forming a double-stranded RNA region, which may be over the entire length of the first and second or third and fourth region, and at least between the mentioned about 19-nucleotide stretches, and wherein the double-stranded RNA regions formed between the first and second region and the double-stranded RNA region formed between the third and fourth region are about equal in size. The population of dsRNA-containing organisms is then analyzed for the down-regulation of the expression of the reporter gene and dsRNA-containing organisms in which the downregulation of the expression of the reporter gene corresponds to the desired degree of downregulation are selected and isolated.

The correspondence in the degree of silencing of two or more genes induced by the presence of dsRNA that comprises the double-stranded complementary sense and antisense RNA regions for the two or more target genes, may also be used to modulate the degree of silencing of those target genes. The correspondence in the degree of silencing of two or more genes is dependent on the relative size of the double-stranded complementary sense and antisense RNA regions. Consequently, the degree of silencing of one particular gene may be modulated by including in a dsRNA molecule comprising complementary sense and antisense RNA regions (designed to silence the expression of a particular target gene or genes) an excess of unrelated complementary sequences, also capable of forming a double-stranded RNA by base-pairing.

Without restricting the invention to a particular mode of action, it is thought that the enzyme in eukaryotic cells that is responsible for generating the small RNA molecules of about 21 nt from the dsRNA (such as DICER in Drosophila) may be saturated by including into the dsRNA an excess of dsRNA sequences (i.e, complementary RNA molecules), the sequence of which is unrelated to the nucleotide sequence of the target gene or genes to be silenced.

Thus in one embodiment of the invention, a method is provided for modulating the reduction of the expression of a target gene in a eukaryotic organism, comprising the steps of:

providing a eukaryotic cell with a dsRNA comprising a first region, a second region, a third region and a fourth region, wherein

the first region comprises a nucleotide sequence of at least about 19 consecutive nucleotides having at least about 94% sequence identity to about 19 consecutive nucleotides from the sense nucleotide sequence of the target gene;

the second region comprises a nucleotide sequence of at least about 19 consecutive nucleotides having at least about 94% sequence identity to the complement of about 19 consecutive nucleotides from the sense nucleotide sequence of the target gene;

the first region and second region are capable of forming a double-stranded RNA region which may be over the entire length of the first and second region, and at least between the mentioned stretches of about 19 nucleotides; and

the third region and fourth region comprise complementary nucleotide seqeunces unrelated to the nucleotide sequence of the target gene and are capable of forming a double-stranded RNA.

The size of the double-stranded RNA capable of being formed by base pairing between the third and fourth region may be equal to or larger than the size of the double-stranded RNA capable of being formed by base pairing between the first and the second region. The size of the third/fourth region may also be smaller than the size of the first/second region, depending on the desired degree of modulation of the silencing.

The first and second regions may be capable of forming a double-stranded RNA region at the same time as when the third and fourth regions are forming a double-stranded stranded RNA. In addition, the target gene may be an endogenous gene of the eukaryotic cell or a transgene, stably integrated into the genome of the eukaryotic cells.

As used herein, a sequence is said to be “unrelated” to a target gene when the overall sequence identity between the unrelated sequence and the target sequence is less than about 50%, or less than about 45%, or less than about 35%. The unrelated sequence will not have a nucleotide sequence having at least about 94% sequence identity with a stretch of about 19 consecutive nucleotides of the target gene or with the complement therof.

The natural variation in downregulation of the expression of a target gene occuring between different lines of a eukaryotic organism comprising the same dsRNA molecule will be shifted towards the lower end of the spectrum of gene silencing. This is due to inclusion of extra dsRNA nucleotide sequences unrelated to the target gene, which are operably linked to the dsRNA formed by the first and second RNA region.

The ratio of the size of the double-stranded RNA capable of being formed by base-pairing between the third and fourth region to the size of the double-stranded RNA capable of being formed by base-pairing between the first and the second region may vary from 0.1 to 20, alternatively from 1 to 10. The greater the mentioned ratio, the more organisms in a population of eukaryotic organisms comprising that dsRNA will have a lower degree of gene silencing (i.e., a higher level of expression of the target gene).

The about 19 nucleotides of the first or second RNA region, which are at least about 94% identical or complementary to a nucleotide sequence of about 19 consecutive nucleotides of the target gene, may be comprised within a larger RNA molecule. Such a longer RNA molecule will have a nucleotide sequence varying between as little as about 19 bp to a length equal to the size of the target gene with a varying overall degree of sequence identity.

The mentioned sense or antisense nucleotide regions may thus be about 50 nt, 100 nt, 200 nt, 300 nt, 500 nt, 1000 nt, 2000 nt or even about 5000 nt or larger in length, each having an overall sequence identity of respectively about 40%, 50%, 60%, 70%, 80%, 90% or 100%. The longer the sequence, the less stringent the requirement for the overall sequence identity.

The first, second, third and fourth regions each may be separated by a spacer region having a nucleotide sequence which is unrelated to the nucleotide sequence of either the target or the reporter gene.

Conveniently, the dsRNA molecules may be introduced into the eukaryotic cells or organisms by transcription from a chimeric gene comprising a promoter or promoter region capable of transcribing a DNA region, which when transcribed yields the dsRNA according to this aspect of the invention. It is understood that different embodiments concerning suitable promoters, etc. mentioned in relation to the first aspect of the invention can also be applied here.

The invention also aims at providing dsRNA or chimeric genes capable of producing such dsRNA molecules according to the second aspect of the invention, as well as eukaryotic organisms comprising such dsRNA or chimeric genes.

The inverted repeat part of the chimeric genes of the invention can be conventionally constructed by recombinational cloning, using the means and methods described in U.S. patent applications Nos. 60/244,067 and 60/333,743 (incorporated herein by reference) or PCT publication WO02/059294. Vectors according to U.S. patent applications No. 60/244,067 or U.S. No. 60/333,743 may be modified to include a third and fourth region capable of forming a double-stranded RNA gene, wherein the third and fourth regions are capable of downregulating the expression of a marker gene such as EIN2, PHYB, FLC and CHS.

The chimeric genes encoding dsRNA according to the invention may comprise an intron, which may be in the region between the second and third region, in order to increase efficiency (as described in WO99/53050). As used herein, an “intron” or intervening sequence is used to refer to a DNA region within a larger transcribed region, which is transcribed in the nucleus to yield an RNA region that is part of a larger RNA; however, the RNA corresponding to the intron sequence is removed from the larger RNA when transferred to the cytoplasm. The corresponding RNA is also referred to as an intron or intervening sequence. Intron sequences are flanked by splice sites, and synthetic introns may be made by joining appropriate splice sites to any sequence having an appropriate branching point. Examples of introns include the pdk2 intron, catalase intron from Castor bean, Delta 12 desaturase intron from cotton, Delta 12 desaturase intron from Arabidopsis, ubiquitin intron from maize, and the intron from SV40.

In one embodiment of the invention, combining the first and second aspect of the invention, a dsRNA molecule may be provided to a eukaryotic cell for monitoring the downregulation of the expression of a target gene by analyzing the downregulation of a second or reporter gene, as described herein. In such an embodiment, the ratio between the size of the dsRNA having sequence to the target gene or its complement and the size of the dsRNA having sequence to the second gene or its complement is between 2 and 10. It is expected that in this way, by identifying the organisms wherein the expression of second gene is effectively downregulated, a population of organisms wherein the expression of the target gene is severely downregulated will be identified.

The methods and means described herein can be applied to any eukaryotic organism in which gene silencing takes place. Such organisms include, but are not limited to: plants (such as corn, cotton, Arabidopsis, rice, vegetables, soybeans, tobacco, trees etc.); invertebrate animals (such as insects, shellfish, molluscs, crustaceans such as crabs, lobsters and prawns); vertebrate animals (fish, birds, mammals, humans); yeast; and fungi amongst others.

The following non-limiting Examples describe method and means for monitoring dsRNA mediated silencing of the expression of a target gene through analysis of a second gene, as well as methods and means for modulating the degree of dsRNA mediated gene silencing in eukaryotic cells.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

Throughout the description and Examples, reference is made to the following sequences:

SEQ ID NO: 1: oligonucleotide primer for PCR reaction of the CHS/FLC fragment.

SEQ ID NO: 2: oligonucleotide primer for PCR reaction of the CHS/FLC fragment.

SEQ ID NO: 3: oligonucleotide primer for PCR reaction of the CHS/FLC fragment.

SEQ ID NO: 4: oligonucleotide primer for PCR reaction of the CHS/FLC fragment.

SEQ ID NO: 5: attB1 recombination site.

SEQ ID NO: 6: attB2 recombination site.

SEQ ID NO: 7: pHELLSGATE 8.

### EXAMPLES

### Example 1

### An ihpRNA Construct Targeting Two Genes Gives Stoichiometric Silencing

Construction of an ihpRNA Construct Targeting Two Genes.

A chimeric gene encoding an ihpRNA construct targeting two distinct genes was constructed. This chimeric gene comprised 300 nt of the Flower Locus C (FLC) gene (Genbank accession AF116527, bases 620-920) followed by 300 nt of the Chalcone synthase (CHS) gene (Genbank accession Y18603, bases 147-532) in sense orientation, and a nucleotide sequence complementary to the mentioned 300 nt of CHS followed by the complementary nucleotide sequence to the mentioned 300 nt of FLC.

The CHS/FLC insert was generated by amplifying a 300 bp fragment of FLC with the oligonucleotide primers having the following sequences:

5′ CTCGAGTCTAGAGGAGCAGMGCTGAGATGGAG 3′ (SEQ ID NO: 1) (primer 138)

5′ CTGCAGGMTAAGGTACAMGTTCATC 3′ (SEQ ID NO: 2) (primer 136)

and cloning the resulting product into pGEM T-easy (Promega, Madison, Wis.).

The CHS fragment was amplified with the oligonucleotide primers having the following sequences:

5′ CTGCAGGCACTGCTMCCCTGAGAAAC 3′ (SEQ ID NO: 3) (primer 133)

5′ GGTACCTTGACTTGGGCTGGCCCCACT 3′ (SEQ ID NO: 4) (primer 143)

and also cloned into pGEM T-easy.

A 300 bp fragment was excised from pGEM T-easy CHS containing the CHS fragment and inserted into the PstI site of pGEM T-easy FLC. The resulting CHS/FLC chimeric insert was amplified using primers with the same gene-specific sequence as 138 and 143 but modified to comprise also the nucleotide sequence encoding the recombination sites attB1 (GGGGACMGTTTGTACAAAAMGCAGGCT; SEQ ID NO: 5) and attB2 (GGGACCACTTTGTACMGAAAGCTGGGT; SEQ ID NO: 6), respectively at their 5′ ends with F1 Taq DNA polymerase (Fisher Biotec, Subiaco, WA, Australia) using the manufaturer's protocol.

PCR products were precipitated by adding 3 volumes TE and two volumes 30% (w/v) PEG 3000, 30 mM MgCl₂ and centrifuging at 13000×g for 15 minutes. Recombination reaction of PCR products with pDONR201 (Invitrogen, Groningen, The Netherlands) was carried out in a total volume of 10 μL with 2 μL BP clonase buffer (Invitrogen), 1-2 μL PCR product 150 ng plasmid vector and 2 μL BP clonase (Invitrogen). The reaction was incubated at room temperature (25° C.) for 1 h to overnight. After the incubation, 1 μL proteinase K (2 μg/μL; Invitrogen) was added and incubated for 10 min at 37° C. 1-2 μL of the mix was used to transform E. coli DH5α, and colonies were selected on the appropriate antibiotics. Clones were checked either by digestion of DNA minipreps or PCR.

Recombination reactions from pDONR201 clones to pHellsgate 8 were carried out in 10 μL total volume with 2 μL LR clonase buffer (Invitrogen), 2 mL pDONR201 clone (approximately 150 ng), 300 ng pHellsgate 8 and 2 μL LR clonase (Invitrogen). The reaction was incubated overnight at room temperature, the proteinase treated, and used to transform DH5α as for the BP clonase reaction.

pHELLSGATE 8 is a vector suitable for recombinational cloning in such a way that an inverted repeat of the insert DNA of interest is generated. The construction of pHELLSGATE 8, and its use for recombination cloning have been described in PCT application PCT/AU02/00073, U.S. No. 60/264,067, and U.S. priority application U.S. No. 60/333,743 (all incorporated herein by reference). pHELLSGATE 8 comprises the following DNA elements:

a right border sequence of Agrobacterium tumefaciens T-DNA region;

a CaMV 35S promoter region;

an attR1 recombination site;

a ccdB selection marker gene from E. coli;

an attR2 recombination site;

a pdk2 (Flavineria trinerva pyruvate orthophosphate dikinase intron 2) intron sequence;

a attR2 recombination site;

a ccdB selection marker gene;

an attR1 recombination site;

a terminator region from Agrobacterium tumefaciens octopine synthase gene (ocs);

a chimeric nptII gene flanked by a nopaline synthase gene (nos) promoter and nos terminator;

a left border Agrobacterium tumefaciens T-DNA region; and

an origin of replication for E. coli and a streptomycin-resistance gene.

The complete sequence of pHELLSGATE 8 is represented in SEQ ID NO: 7.

Upon recombinational cloning of the CHS/FLC, PCR amplified insert as described above, a chimeric gene is thus generated comprising:

a CaMV35S promoter region;

a nucleotide sequence corresponding to bases 620-920 of FLC;

a nucleotide sequence corresponding to bases 147-532 of CHS;

a pdk2 intron;

a nucleotide sequence complementary to bases 147-532 of CHS;

a nucleotide sequence complementary to bases 620-920 of FLC; and

an ocs terminator region.

This chimeric gene is located between Agrobacterium T-DNA border sequences, together with a plant-expressible selectable marker gene. The vector can thus be introduced into Agrobacteria comprising the required helper functions, and used to transform plant cells.

Plant Transformation.

Transformation of Arabidopsis C24 ecotype was via the floral dip method (Clough and Bent, 1998). Plants were selected on agar solidified MS media supplemented with 100 mg/l timentin and 50 mg/l kanamycin.

Phenotypic Analysis Methods for Plants Wherein FLC or CHS Genes are Silenced.

T1 FLC ihpRNA plants were scored by transferring to MS plates and scoring days to flower or rosette leaves at flowering compared to C24 wild type plants and flc mutant lines. Plant lines wherein the FLC gene expression is reduced or eliminated flower earlier than wild type C24 ecotype. A block in anthocyanin biosynthesis by reducing expression of chalcone synthase gene (CHS) leads to the accumulation of malonyl-CoA, which increases fluorescence of seed observed under UV light.

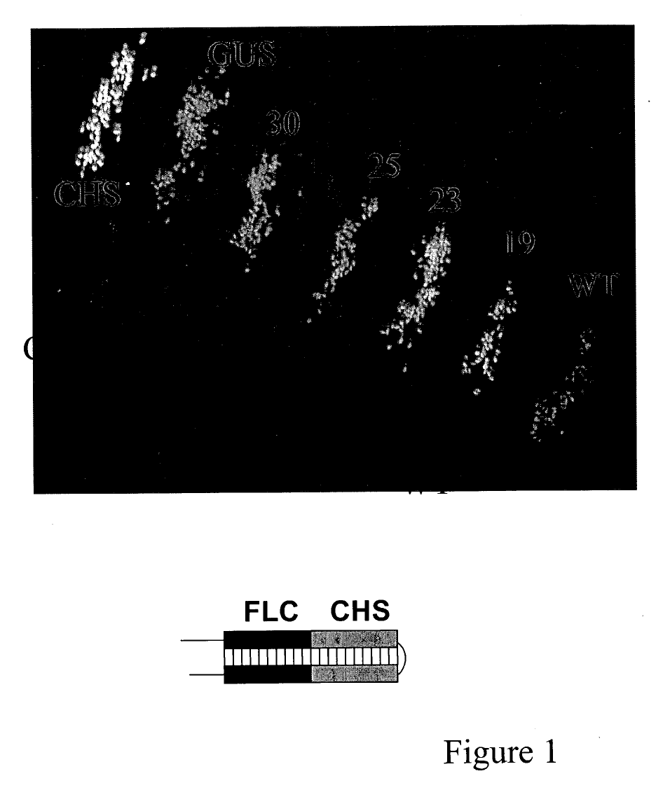

All of the T1 plants transformed with the iph CHS/FLC construct flowered earlier than wild type C24 (36 days) and later than the transposon-tagged flc-13 line (17 days; Sheldon et al, 1999). The seeds from four ihp CHS/FLC plants that flowered at different times were tested for fluorescence (FIG. 1) compared to wildtype C24, a 35S::GUS line and a homozygous ihpRNA-silenced CHS line (Wesley et al, 2001). The relative concentrations of the different intermediates in the chalcone synthase pathway were also determined by HPLC analysis.

The CHS ihpRNA line shows strong fluorescence reflecting the accumulation of malonyl-CoA in this line while the wildtype C24 and the 35S::GUS lines do not show fluorescence under UV light. The four ihp CHS/FLC lines examined all show some fluorescence with the earliest flowering lines showing the strongest fluorescence.

### Example 2

### Modulation of the Degree of Silencing of CHS Gene by Dilution With Extra Non-Target Sequences in the Stem of the iphRNA.

To test the influence of the inclusion of extra nucleotides, unrelated to the target DNA sequences, in the ihpRNA or dsRNA molecules on the degree of silencing of a target gene, a number of chimeric genes were generated. Each chimeric gene comprised 100 bp from the CHS nucleotide sequence (corresponding to Genbank accession Y18603) both in anti-sense and sense orientation (referred to as CHSₐₙₜᵢₛₑₙₛₑ and CHSₛₑₙₛₑ, respectively).

Progressively more nucleotide sequences (from 100 bp to 900 bp; referred to as Extra-NNN-ntₛₑₙₛₑ or Extra-NNN-ntₐₙₜᵢₛₑₙₛₑ) unrelated to the CHS gene were inserted both in antisense and sense orientation. The unrelated nucleotide sequences were derived from the FLC gene (corresponding to Genbank accession Nr AF116527).

The different CHS/Extra nucleotide constructs were PCR-amplified with attB1 and attB2 extended primers and introduced into pHELLSGATE 8 as described in Example 1. The resulting plasmids thus contain the following chimeric dsRNA genes:

1. <CaMV35S-CHSₛₑₙₛₑ-Extra_{—}100_ntₛₑₙₛₑ-pdk2intron-Extra_{—}100_ntₐₙₜᵢₛₑₙₛₑ-CHSₐₙₜᵢₛₑₙₛₑ-ocs terminator>; or

2. <CaMV35S-CHSₛₑₙₛₑ-Extra_{—}300_ntₛₑₙₛₑ-pdk2intron-Extra_{—}300_ntₐₙₜᵢₛₑₙₛₑ-CHSₐₙₜᵢₛₑₙₛₑ-ocs terminator>; or

3. <CaMV35S-CHSₛₑₙₛₑ-Extra_{—}500_ntₛₑₙₛₑ-pdk2intron-Extra_{—}500_ntₐₙₜᵢₛₑₙₛₑ-CHSₐₙₜᵢₛₑₙₛₑ-ocs terminator>; or

4. <CaMV35S-CHSₛₑₙₛₑ-Extra_{—}900_ntₛₑₙₛₑ-pdk2intron-Extra_{—}900_ntₐₙₜᵢₛₑₙₛₑ-CHSₐₙₜᵢₛₑₙₛₑ-ocs terminator>.

Transformed Arabidopsis plant lines comprising one of the four above mentioned chimeric genes were generated as described in Example 1, and fluorescence under UV light to monitor silencing of the CHS gene was scored as described in Example 1 for seeds of plants of each line. Independent transgenic lines were classified according to the degree of fluorescence. The results are summarized in Table 1.

TABLE 1
Number of independent transgenic lines showing different classes of
fluorescence.

Fluorescence level

| | − | + | ++ | +++ |
|---|---|---|---|---|
| 100 nt CHS + 100 extrant | 12 | 0 | 20 | 0 |
| 100 nt CHS + 300 extrant | 11 | 18 | 11 | 1 |
| 100 nt CHS + 500 extrant | 5 | 7 | 13 | 0 |
| 100 nt CHS + 900 extrant | 18 | 8 | 3 | 0 |

From these results, it is clear that the higher the ratio of extra nucleotide sequences versus target sequences in the ihpRNA construct, the higher the proportion of transgenic plant lines obtained with a lower degree of fluorescence, i.e., with a lower degree of silencing of the expression of the CHS target gene.

Inclusion of extra, unrelated, sequences in ihpRNA or dsRNA effectively shifts the distribution of silencing in the population of transgenic lines towards the lower end of the spectrum of gene-silencing.

### Example 3

### Northern Analysis of Transgenic Plant Lines Comprising a Chimeric Gene Encoding Simultaneously dsRNA Regions Targeted Towards FLC and CHS.

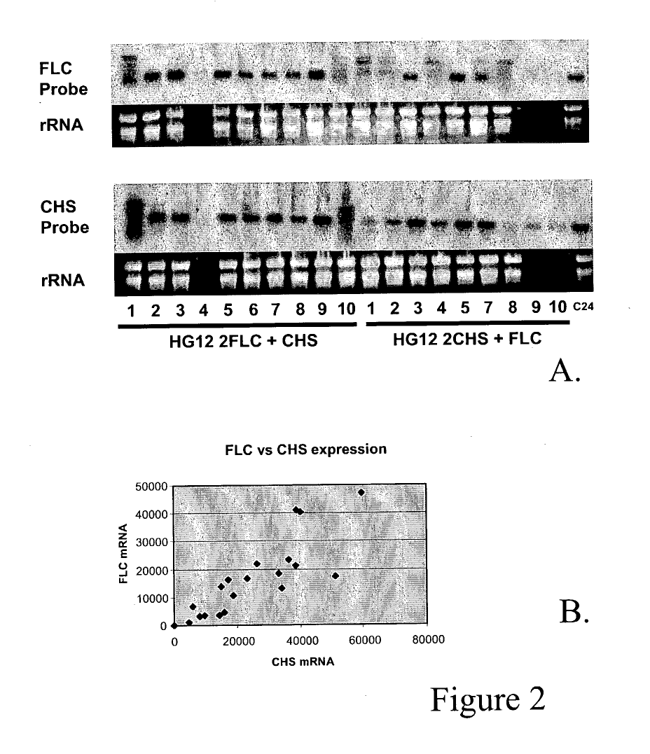

RNA was prepared from different Arabidopsis plant lines comprising two FLC-CHS hairpin constructs. One construct was a modified Hellsgate 12 vector (see WO02/059294) with a FLC hairpin next to the intron with a CHS fragment inserted in the attR sites. The other construct was a modified Hellsgate 12 with a CHS hairpin next to the intron with an FLC fragment inserted into the attR sites. Two identical gels were run, and blotted and probed with either FLC or CHS antisense RNA probes. The blots were RNase-treated to ensure the signals were specific. The resulting autoradiogram is represented in FIG. 2A. Estimates of the amount of hybridization for the different probes and lines are summarized in Table 2 and graphically represented in FIG. 2B. There appears be a good linear direct relation between the amount of FLC and CHS signals in each line indicating that where one gene is silenced the other gene is also silenced to the same degree.

TABLE 2
Estimates of hybridization signals in different plants using either
FLC or CHS antisense probes.

| Plant | FLC mRNA | CHS mRNA |
|---|---|---|
| HG12 2FLC + 2CHS #1 | 13867 | 15154 |
| HG12 2FLC + 2CHS #2 | 21962 | 26467 |
| HG12 2FLC + 2CHS #3 | 40434 | 40096 |
| HG12 2FLC + 2CHS #4 | 0 | 0 |
| HG12 2FLC + 2CHS #5 | 41122 | 38987 |
| HG12 2FLC + 2CHS #6 | 16609 | 23091 |
| HG12 2FLC + 2CHS #7 | 18417 | 3357 |
| HG12 2FLC + 2CHS #8 | 16463 | 17400 |
| HG12 2FLC + 2CHS #9 | 47210 | 59680 |
| HG12 2FLC + 2CHS #10 | 10792 | 18879 |
| HG12 2FLC + 2FLC #1 | 3399 | 9694 |
| HG12 2FLC + 2FLC #2 | 3692 | 14317 |
| HG12 2FLC + 2FLC #3 | 1725 | 51004 |
| HG12 2FLC + 2FLC #4 | 4620 | 16067 |
| HG12 2FLC + 2FLC #5 | 21826 | 38643 |
| HG12 2FLC + 2FLC #7 | 13288 | 34043 |
| HG12 2FLC + 2FLC #8 | 6716 | 5949 |
| HG12 2FLC + 2FLC #9 | 3027 | 8036 |
| HG12 2FLC + 2FLC #10 | 1021 | 4677 |
| C24 wildtype | 23376 | 36279 |

### REFERENCES

An et al., 1996 The Plant Cell 8, 15-30

Butler and Chamberlin, 1982 J. Biol. Chem., 257: 5772-5778

Dunn and Studier 1983 J. Mol. Biol. 166: 477-535

Dunn et al., 1971 Nature New Biology, 230: 94-96

Fire et al., 1998 Nature 391, 806-811

Hamilton et al. 1998 Plant J. 15: 737-746

Hapster et al., 1988 Mol. Gen. Genet. 212, 182-190

Hausmann, 1976 Current Topics in Microbiology and Immunology, 75: 77-109

Hudspeth et al., 1989 Plant Mol Biol 12: 579-589

Keil et al., 1989 EMBO J. 8: 1323-1330

Keller et al., 1988 EMBO J. 7: 3625-3633

Keller et al.,1989 Genes Devel. 3: 1639-1646

Klement et al., 1990 J. Mol. Biol. 215, 21-29

Korsten et al., 1975 J. Gen Virol. 43: 57-73

McGraw et al, 1985 Nucl. Acid Res. 13: 6753-6766

Needleman and Wunsch 1970

Peleman et al., 1989 Gene 84: 359-369

Smith et al., 2000 Nature 407: 319-320

Towle et al., J. Biol. Chem. 250: 1723-1733 (1975)

Waterhouse et al. 1998 Proc. Natl. Acad. Sci. USA 95: 13959-13964

We claim:

1. A method for monitoring the reduction of the expression of a target gene in a cell of a eukaryotic organism, the method comprising the steps of:
providing a eukaryotic cell with a dsRNA comprising a first region, a second region, a third region and a fourth region, wherein
the first region comprises a nucleotide sequence of at least about 19 consecutive nucleotides, which has at least about 94% sequence identity to about 19 consecutive nucleotides from a sense nucleotide region of the target gene;
the second region comprises a nucleotide sequence of at least about 19 consecutive nucleotides, which has at least about 94% sequence identity to a nucleotide sequence complementary to about 19 consecutive nucleotides from the sense nucleotide region of the target gene;
the first region and the second region are capable of forming a double-stranded RNA region;
the third region comprises a nucleotide sequence of at least about 19 consecutive nucleotides, which has at least about 94% sequence identity to about 19 consecutive nucleotides from a sense nucleotide region of a second gene present in the eukaryotic cell and which is different from the target gene;
the fourth region comprises a nucleotide sequence of at least about 19 consecutive nucleotides, which has having at least about 94% sequence identity to the complement of the about 19 consecutive nucleotides from the sense nucleotide region of the second gene; and
the third and fourth region are capable of forming a double-stranded RNA region; and
monitoring the reduction of the expression of the target gene by analyzing the reduction in expression of the second gene.

2. The method of claim 1, wherein the eukaryotic organism is a plant.

3. The method of claim 1, wherein the eukaryotic organism is an animal.

4. The method of claim 1, wherein the eukaryotic organism is a yeast, fungus or mold.

5. The method of claim 2, wherein the eukaryotic organism is selected from the group consisting of cotton, potato, corn, wheat, rice, sugar cane, oilseed rape, Arabidopsis, sugarbeet, tobacco and soybean.

6. The method of claim 3, wherein the eukaryotic organism is selected from the group consisting of insects, shellfish, molluscs, crustaceans, crabs, lobsters, prawns, fish, birds, mammals and humans.

7. The method of claim 1, wherein the second gene is an endogenous gene present in the eukaryotic cell.

8. The method of claim 1, wherein the second gene is a transgene stably integrated into the genome of the eukaryotic cell.

9. The method of claim 2, wherein the second gene is selected from the group consisting of PDS, EIN2, FLC and PhyB.

10. The method of claim 1, wherein the eukaryotic cell comprises a functionally expressed GUS or a GFP gene, and the second gene is a GUS or GFP gene.

11. The method of claim 1, wherein the first and second region, and the third and fourth region are about 300 nt in length.

12. The method of claim 1, wherein the dsRNA is transcribed from a chimeric gene comprised within cells of the eukaryotic organism, and wherein the chimeric gene comprises the following operably-linked elements:
a promoter region which functions in the eukaryotic cell;
a DNA region which when transcribed yields the dsRNA molecule; and
a transcription termination and polyadenylation region that functions in the eukaryotic organism.

13. The method according to claim 12, wherein the chimeric gene is stably integrated into the genome of cells of the eukaryotic organism.

14. A dsRNA comprising a first region, a second region, a third region and a fourth region, wherein
the first region comprises a nucleotide sequence of at least about 19 consecutive nucleotides, which has at least about 94% sequence identity to about 19 consecutive nucleotides from a sense nucleotide region of the target gene;
the second region comprises a nucleotide sequence of at least about 19 consecutive nucleotides, which has at least about 94% sequence identity to a nucleotide sequence complementary to about 19 consecutive nucleotides from the sense nucleotide region of the target gene;
the first region and the second region are capable of forming a double-stranded RNA region;
the third region comprises a nucleotide sequence of at least about 19 consecutive nucleotides, which has at least about 94% sequence identity to about 19 consecutive nucleotides from a sense nucleotide region of a second gene present in the eukaryotic cell and which is different from the target gene;
the fourth region comprises a nucleotide sequence of at least about 19 consecutive nucleotides, which has at least about 94% sequence identity to the complement of the about 19 consecutive nucleotides from the sense nucleotide region of the second gene; and
the third and fourth region are capable of forming a double-stranded RNA region.

15. A DNA molecule for measuring the reduction of expression of a target gene in a cell of a eukaryotic organism, comprising the following operably linked elements:
a promoter region that functions in the eukaryotic cell;
a DNA region which when transcribed yields a dsRNA molecule, wherein the dsRNA comprises a first, second, third and fourth region; and
a transcription termination and polyadenylation region that functions in the eukaryotic organism; wherein
the first region comprises a nucleotide sequence of at least about 19 consecutive nucleotides, which has at least about 94% sequence identity to about 19 consecutive nucleotides from a sense nucleotide region of the target gene;
the second region comprises a nucleotide sequence of at least about 19 consecutive nucleotides, which has at least about 94% sequence identity to a nucleotide sequence complementary to about 19 consecutive nucleotides from the sense nucleotide region of the target gene;
the first region and the second region are capable of forming a double-stranded RNA region;
the third region comprises a nucleotide sequence of at least about 19 consecutive nucleotides, which has at least about 94% sequence identity to about 19 consecutive nucleotides from a sense nucleotide region of a second gene present in the eukaryotic cell, and which is different from the target gene;
the fourth region comprises a nucleotide sequence of at least about 19 consecutive nucleotides, which has at least about 94% sequence identity to the complement of the about 19 consecutive nucleotides from the sense nucleotide region of the second gene;
the third and fourth region are capable of forming a double-stranded RNA region; and
wherein the second gene is an endogenous gene of the eukaryotic organism or a transgene stably integrated into the genome of cells of the eukaryotic organism.

16. A eukaryotic organism comprising an RNA molecule according to claim 14

17. A eukaryotic organism comprising a DNA molecule according to claim 15.

18. The eukaryotic organism according to claim 16, which is a plant.

19. The eukaryotic organism according to claim 17, which is a plant.

20. The eukaryotic organism according to claim 16, which is an animal.

21. The eukaryotic organism according to claim 17, which is a yeast, fungus or mold.

22. A method for identifying, within a population of dsRNA-mediated gene-silenced eukaryotic organisms, those organisms with the desired degree of silencing of a target gene, comprising:
providing cells of the eukaryotic organisms with a dsRNA comprising a first region, a second region, a third region and a fourth region, wherein
the first region comprises a nucleotide sequence of at least about 19 consecutive nucleotides, which has at least about 94% sequence identity to about 19 consecutive nucleotides from a sense nucleotide region of the target gene;
the second region comprises a nucleotide sequence of at least about 19 consecutive nucleotides, which has at least about 94% sequence identity to a nucleotide sequence complementary to about 19 consecutive nucleotides from the sense nucleotide region of the target gene; the first region and the second region are capable of forming a double-stranded RNA region;
the third region comprises a nucleotide sequence of at least about 19 consecutive nucleotides, which has at least about 94% sequence identity to about 19 consecutive nucleotides from a sense nucleotide region of a second gene present in the eukaryotic cells, and which is different from the target gene;
the fourth region comprises a nucleotide sequence of at least about 19 consecutive nucleotides, which has at least about 94% sequence identity to the complement of the about 19 consecutive nucleotides from the sense nucleotide region of the second gene;
the third and fourth region are capable of forming a double-stranded RNA region; and
identifying the organism with the desired degree of silencing of the target gene, by selecting the organisms with the desired degree of silencing of the second gene.

23. A method for modulating the reduction of the expression of a target gene in a cell of a eukaryotic organism, comprising the steps of:
providing the cell of the eukaryotic organism with a dsRNA comprising a first region, a second region, a third region and a fourth region, wherein
the first region comprises a nucleotide sequence of at least about 19 consecutive nucleotides, which has at least about 94% sequence identity to about 19 consecutive nucleotides from the sense nucleotide sequence of the target gene;
the second region comprises a nucleotide sequence of at least about 19 consecutive nucleotides, which has at least about 94% sequence identity to the complement of about 19 consecutive nucleotides from the sense nucleotide sequence of the target gene;
the first region and the second region are capable of forming a double-stranded RNA region;
the third region and the fourth region comprise complementary nucleotide sequences which have a sequence identity of less than 50% to the nucleotide sequence of the target gene, and which are capable of forming a double-stranded RNA; and
wherein the target gene is an endogenous gene in the eukaryotic cell or a transgene stably integrated in the genome of the eukaryotic cell.

24. The method of claim 23, wherein the size of the double-stranded RNA capable of being formed by base-pairing between the third and fourth region is equal in size to, or larger than, the double-stranded RNA capable of being formed by base-pairing between the first and the second region.

25. The method of claim 23, wherein the eukaryotic organism is a plant.

26. The method of claim 23, wherein the eukaryotic organism is an animal.

27. The method of claim 23, wherein the eukaryotic organism is a yeast, fungus or mold.

28. The method of claim 25, wherein the plant is selected from the group of cotton, potato, corn, wheat, rice, sugar cane, oilseed rape, Arabidopsis, sugarbeet, tobacco or soybean.

29. The method of claim 26, wherein the animal is selected from the group consisting of insects, shellfish, molluscs, crustaceans, crabs, lobsters, prawns, fish, birds, mammals and humans.

30. An RNA molecule for modulating the expression of a target gene in a cell of a eukaryotic organism, comprising a first region, a second region, a third region and a fourth region, wherein
the first region comprises a nucleotide sequence of at least about 19 consecutive nucleotides, which has at least about 94% sequence identity to about 19 consecutive nucleotides from the sense nucleotide sequence of the target gene;
the second region comprises a nucleotide sequence of at least about 19 consecutive nucleotides, which has at least about 94% sequence identity to the complement of about 19 consecutive nucleotides from the sense nucleotide sequence of the target gene;
the first region and the second region are capable of forming a double-stranded RNA region;
the third region and the fourth region comprising complementary nucleotide sequences that have a sequence identity of less than 50% to the nucleotide sequence of the target gene, and that are capable of forming a double-stranded RNA; and
the target gene is an endogenous gene in the eukaryotic cell or a transgene, stably integrated in the genome of the eukaryotic cell.

31. A DNA molecule capable of producing a dsRNA molecule according to claim 31, comprising a DNA region that, when transcribed, yields the dsRNA molecule, wherein the DNA region is operably linked to a promoter and a transcription termination and polyadenylation signal.

32. A eukaryotic organism comprising a DNA molecule according to claim 31.

33. A eukaryotic organism comprising an RNA molecule according to claim 30.
